(19) United States
(12) Patent Application Publication
(10) Pub. No.: US 2007/0148636 A1
(43) Pub. Date: Jun. 28, 2007

(54) Method, compositions and kits for preparation of nucleic acids

(76) Inventors: Min-sun Song, San Francisco, CA (US); Diane D. Ilsley, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/317,557

(22) Filed: Dec. 23, 2005

(51) Int. Cl. C12Q 1/70; C12Q 1/68; C12N 15/74

(52) U.S. Cl. 435/5; 435/6; 435/472

(57) ABSTRACT

Methods, compositions and kits are provided for labeling, copying and/or amplifying nucleic acids. The methods, compositions and kit can be used for a variety of applications, for example, genome-wide scanning applications such as CGH or location analysis.

### BACKGROUND

Comparative genomic hybridization (CGH) is an approach that has been employed to detect the presence of and identify the location of amplified or deleted sequences. In one implementation of CGH, genomic DNA is isolated from reference cells (e.g., cells with a known genomic content or copy number at at least one locus) as well as from test cells (e.g., tumor cells). The relative amount of DNA in the test sample vs. the reference sample can be used to identify the occurrence of deletions and/or duplications in nucleic acids of the test sample compared to the reference sample, which can be used in certain cases to diagnose or predict the risk of a pathology such as cancer.

The ratio of DNA in the test sample vs. the reference sample can be evaluated in a number of different ways. For example, the two samples can be simultaneously hybridized in situ to metaphase chromosomes of a reference cell. Chromosomal regions in the test cells that are at increased or decreased copy number can be identified by detecting regions where the ratio of signal from the two DNAs is altered. For example, those regions that have been decreased in copy number in the test cells show relatively lower signal from the test DNA than the reference compared to other regions of the genome. Regions that have been increased in copy number in the test cells show relatively higher signal from the test DNA.

In another variation of CGH approach, the immobilized chromosome element is replaced with a collection of solid support-bound nucleic acids, e.g., an array of BAC (bacterial artificial chromosome) clones, cDNAs, or oligonucleotides. “Array CGH” (aCGH) offers benefits over immobilized chromosome approaches, including a higher resolution, as defined by the ability of the assay to localize chromosomal alterations to specific areas of the genome.

CGH measurements need to distinguish genomic lesions such as homozygous deletions, low-level amplification and single copy losses using complex targets derived from genomic DNA (gDNA). In making such measurements, it is crucial to minimize variation among signals arising from DNA having the same copy number.

In an aCGH experiment, DNA template (e.g., amplified DNA or genomic DNA) from a sample is often digested into fragments with restriction enzymes, denatured into single strands and labeled using a DNA polymerase I (pol I)-type enzyme such as Klenow (DNA pol I large fragment) in the presence of a label such as a fluorescent dye (e.g., Cy3 or Cy5). In a processive labeling protocol, replication is initiated at a random site, by transient annealing of short random unlabeled oligomers (6-10 mers), which are extended by the polymerase in the presence of labeled nucleotides. Typically the test sample is labeled with one type of label, (e.g., such as Cy3) and the reference sample is labeled with a different type of label (e.g., such as Cy5). The two samples are mixed and the mixture is allowed to hybridize for a period of time with an array containing probes complementary to target fragments of interest. The ratio of the signals measured for the two labels from each probe spot is used to deduce the ratio of copy numbers of the targets of interest in the original sample and reference DNA. Generally, contacting the two samples to a single array (vs. contacting each sample to two identical arrays) is less sensitive to array-to-array variations in probe features and hybridization conditions. However, analysis using a single array may still be subject to systematic bias because of differences between the two labels, arising from different labeling efficiencies and/or from different sensitivity due to fluorophore quenching.

Further, although widely used, Klenow-based labeling using cyanine dyes is associated with number of limitations, either because of the labeling process itself or because of array processing artifacts. These include relatively low signal intensities after hybridization and washes and average signal intensity in the Cy5 dye channel, and dye bias even when the initial template DNA and dye concentrations are identical. Thus, current labeling protocols can contribute to a significant portion of the variation in CGH measurements. In addition, Klenow has relatively low fidelity (approximately 1.3×10⁻⁴) and thus can introduce mutations in a copied template.

Further, the low processivity of Klenow fragment or pol I derivatives often results in very short products (<20 nucleotides) which do not hybridize efficiently. This can result in low signal intensities on arrays and variable representation of genomic DNA in a sample, particularly if the genomic DNA has repetitive sequences or extensive secondary structure. Additionally, Klenow is not particularly suited for use in methods for amplifying DNA templates. Combined with its strand displacement activity and potential tendency to preferentially replicate certain regions of DNA (for example, sequences which lack secondary structure), use of Klenow could result in uneven representation of certain types of sequences in an amplified sample.

The phi29 polymerase has been used in whole genome amplification methods to provide relatively unbiased copying of genomic template DNA. The relative representation of individual loci has been estimated to differ by less than 6-fold compared to unamplified genomic DNA (Hosono, et al., Genome Res. 2003 May;13(5):954-64). Amplification methods relying on phi29 polymerase typically are carried out under isothermal conditions and involve multiple strand displacement amplification since the polymerase is capable of polymerizing >70 kb w/o dissociating from a genomic DNA template.

However, to insure complete coverage and representation of the genome in question, current protocols using phi29 polymerase typically require high quality intact genomic DNA as a starting material (Pollack, et al., Proc Natl Acad Sci USA. 2002;99(20):12963-68). Further, the multiple strand displacement activity of phi29 can cause a high level of branched nucleic acid forms form degraded DNA samples, resulting in non-uniform amplification or labeling of gDNA. Additionally, the use of phi29 polymerase with degraded samples can result in low or insufficient yields of high molecular weight DNA which are suitable for downstream applications such as fluorescent labeling. This is particularly a problem when formalin-fixed paraffin embedded tumor samples are used. Generally, the quality of extracted DNA is very poor and the DNA is often severely degraded.

### SUMMARY

In one embodiment, the invention provides a method of copying non-bacteriophage DNA using a T7-like DNA polymerase. In one aspect, the method comprises contacting a sample of non-phage DNA with a T7-like polymerase in the presence of one or more accessory proteins, such as for example, thioredoxin, a helicase, a primase, a single stranded binding protein, and/or functionally equivalent proteins. In certain aspects, a single protein provides both helicase and primase activities, for example, an accessory protein such as gene 4 protein is provided. Contacting is done in the presence of nucleotides, or modified or derivative forms thereof. In one aspect, the nucleotides are labeled.

In certain aspects, contacting is done in the presence of an oligonucleotide which is complementary to a subsequence of the non-bacteriophage DNA and/or which hybridizes to the subsequence of the non-bacteriophage DNA under stringent hybridization conditions. In another aspect, contacting is done in the presence of a plurality of oligonucleotides. In one aspect, the plurality is selected to bind randomly to subsequences of the non-bacteriophage DNA. In one aspect, the non-bacteriophage comprises eukaryotic DNA, such as mammalian DNA and more particularly human DNA. In another aspect, the DNA is genomic DNA. In a further aspect, contacting is done under conditions in which the non-bacteriophage DNA is copied by the T7-like polymerase.

In another embodiment, the methods are used to copy template DNA to be used for a genome-wide scanning application, such as CGH or location analysis.

In one embodiment, the invention provides methods for copying at least two samples of non-bacteriophage nucleic acids in the presence of first labeled nucleotides and second labeled nucleotides, respectively. In one aspect, the first labeled nucleotides are labeled with Cy3 while the second labeled nucleotides are labeled with Cy5. In another aspect, after the two samples are copied, copied nucleic acids are contacted to a support comprising nucleic acids, e.g., such as a chemical array substrate comprising a plurality of probe nucleic acids. In a further aspect, the first and second samples comprise test and reference nucleic acids, respectively, and the relative ratio of a target sequence in the first and second sample is determined, e.g., to evaluate the relative copy number of the target in the samples, for example, to determine the presence of duplications or deletions of the target in the test sample compared to the reference sample.

In still another embodiment, a sample of nucleic acids is bound to proteins from a cellular source, e.g., via crosslinking, and nucleic acids bound to protein(s) of interest are obtained (e.g., via immunoprecipitation) before or after a fragmentation step (via sonication or by contacting with an endonuclease or a combination thereof). Binding of nucleic acids to the protein of interest is reversed and the fragments are copied using a method as described above. In certain aspects, the fragments bound to the protein of interest and which have been copied are contacted to a chemical array.

In certain aspects, a method according to the invention comprises contacting a sample of non-bacteriophage, non-circular, genomic DNA with a T7-like polymerase in the presence of at least one accessory protein, an oligonucleotide capable of binding to a sequence of the non-bacteriophage genomic DNA, and one or more nucleotides, under conditions wherein the oligonucleotide binds to the sequence of the non-bacteriophage genomic DNA and the T7-like polymerase extends the primer. The at least one accessory protein is selected from the group consisting of a thioredoxin, a helicase, a primase, a single-stranded binding protein, functionally equivalent proteins, and combinations thereof. In certain aspects, contacting is done in the presence of a thioredoxin, a helicase, a primase, and a single-stranded binding protein. In additional aspects, helicase and primase activities are provided in a single protein.

In one aspect, the sample of genomic DNA has the complexity of at least an E. coli genome. In another aspect, the sample of genomic DNA has the complexity of a mammalian genome (e.g., the complexity of a mouse genome, a primate genome, such as a human genome, the genome of a domestic and/or companion animal, etc.).

In certain aspects, contacting occurs in the presence of a plurality of random or degenerate sequence oligonucleotides.

In one aspect, at least one of the one or more nucleotides is labeled.

In one aspect, the contacting occurs under conditions suitable for copying and/or amplifying the genomic DNA in the sample.

In another aspect, contacting occurs under conditions suitable for labeling the genomic DNA in the sample.

In a further aspect, contacting occurs under conditions suitable for labeling copied genomic DNA.

In certain aspects, the method further comprises the step of fragmenting the genomic DNA, e.g., by contacting the genomic DNA with a nuclease.

The method can further include contacting primer extension products to an array.

In certain aspects, the method further comprises performing the method on first and second genomic samples and mixing primer extension products from the first and second samples. In one aspect, the primer extension products from the first and second samples are differentially labeled. In certain aspects, the method further includes the step of determining relative amounts of at least one sequence in the first and second sample. In still other aspects, the method can further comprise performing the method on first and second separate samples and contacting primer extension products to the same array or to at least two arrays comprising at least a subset of identical sequences at features of the arrays.

In one aspect, the genomic sample comprises DNA binding proteins bound thereon and wherein the method comprises a fragmentation step to fragment the genomic DNA at sequences not bound by the DNA binding proteins. In another aspect, the DNA binding proteins are crosslinked to the genomic DNA. In certain aspects, the method further comprises obtaining DNA fragments bound to a DNA binding protein of interest prior to the contacting step, e.g., such as by immunoprecipitation. The fragments so obtained can be contacted to an array. The fragments can be contacted with the T7-like polymerase, at least one accessory protein, nucleotides, and primers before or after contacting to an array of probes for identifying the sequence and/or genomic location of the fragments.

In another aspect, the invention provides a method comprising: contacting a test sample and a reference sample of genomic DNA with a T7-like polymerase in the presence of at least one accessory protein, an oligonucleotide primer capable of binding to a sequence of the genomic DNA, and one or more nucleotides, under conditions wherein the oligonucleotide primer binds to the sequence of the genomic DNA in the test and reference samples and the T7-like polymerase extends the primer, obtaining primer extension products from the first and second samples and contacting primer extension products to the same array or to at least two arrays comprising at least a subset of identical sequences at features of the arrays. The method can further include the step of determining relative amounts of at least one sequence in the test and reference sample.

In still another aspect, invention provides a method comprising: contacting a sample of genomic DNA that comprises DNA binding proteins bound thereon; fragmenting the genomic DNA at sequences not bound by the DNA binding proteins; obtaining DNA fragments bound to a DNA binding protein of interest (e.g., the fragments may be crosslinked to the DNA binding protein and obtained by immunoprecipitation); removing the DNA binding protein of interest from the DNA fragments (e.g., by reversing the crosslinking); contacting the DNA fragments with a T7-like polymerase in the presence of at least one accessory protein, oligonucleotide primers capable of binding to a sequence of a plurality of the fragments, and one or more nucleotides, under conditions wherein the oligonucleotides binds to the sequence of the fragments and T7-like polymerase extends the primer, and contacting primer extension products to an array of nucleic acids. In certain aspects, the method further comprises determining the location and/or sequence of a fragment to which the DNA binding protein of interest binds.

The invention further provides kits. In one aspect, a kit comprises a T7- like polymerase, at least one accessory protein, and a sample of non-bacteriophage, non-circular genomic DNA. For example, the sample can comprise genomic DNA having at least the complexity of E. coli DNA. In certain aspects, the sample comprises genomic DNA having at least the complexity of mammalian DNA.

In one aspect, a kit according to the invention comprises a T7-like polymerase, at least one accessory protein, random or degenerate sequence oligonucleotides, and/or labeled oligonucleotides for binding to a plurality of genomic DNA sequences, or nucleotides, optionally labeled with spectrally distinguishable labels, e.g., such as Cy3 and Cy5, and combinations thereof.

In another aspect, a kit according to the invention comprises a T7-like polymerase, at least one accessory protein, and a deparaffinizing reagent.

In a further aspect, a kit according to the invention comprises a T7- like polymerase, at least one accessory protein, and an antigen-binding molecule specific to a DNA binding protein.

Additionally, kits according to the invention can include one or more arrays for probing primer extension products generated according to methods of the invention.

### DESCRIPTION

Before describing the present invention in detail, it is to be understood that this invention is not limited to specific compositions, method steps, or equipment, as such may vary. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional. feature of the inventive variations described may be set forth and claimed independently, or in combination with any one or more of the features described herein.

Unless defined otherwise below, all technical and scientific terms used herein have the same meaning as commonly understood by one of ordinary skill in the art to which this invention belongs. Still, certain elements are defined herein for the sake of clarity.

All publications (including patents and patent applications) mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “an oligonucleotide primer” can include more than one oligonucleotide primer.

### Definitions

The following definitions are provided for specific terms that are used in the following written description.

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 100 nucleotides in length, while a “polynucleotide” or “nucleic acid” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moieties are stably associated with the substrate surface in the region, such that they do not separate from the region under conditions of using the array, e.g., hybridization and washing and stripping conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

The phrase ” oligonucleotide bound to a surface of a solid support” or “probe bound to a solid support” or a “target bound to a solid support” refers to an oligonucleotide or mimetic thereof, e.g., PNA, LNA or UNA molecule that is immobilized on a surface of a solid substrate, where the substrate can have a variety of configurations, e.g., a sheet, bead, particle, slide, wafer, web, fiber, tube, capillary, microfluidic channel or reservoir, or other structure. In certain embodiments, the collections of oligonucleotide elements employed herein are present on a surface of the same planar support, e.g., in the form of an array. It should be understood that the terms “probe” and “target” are relative terms and that a molecule considered as a probe in certain assays may function as a target in other assays.

“Addressable sets of probes” and analogous terms refer to the multiple known regions of different moieties of known characteristics (e.g., base sequence composition) supported by or intended to be supported by an array surface, such that each location is associated with a moiety of a known characteristic and such that properties of a target moiety can be determined based on the location on the array surface to which the target moiety binds under stringent conditions.

In certain embodiments, an array is contacted with a nucleic acid sample under stringent assay conditions, i.e., conditions that are compatible with producing bound pairs of biopolymers of sufficient affinity to provide for the desired level of specificity in the assay while being less compatible to the formation of binding pairs between binding members of insufficient affinity. Stringent assay conditions are the summation or combination (totality) of both binding conditions and wash conditions for removing unbound molecules from the array.

The term “sample” as used herein relates to a material or mixture of materials, containing one or more components of interest. Samples include, but are not limited to, samples obtained from an organism or from the environment (e.g., a soil sample, water sample, etc.) and may be directly obtained from a source (e.g., such as a biopsy or from a tumor) or indirectly obtained e.g., after culturing and/or one or more processing steps. In one embodiment, samples are a complex mixture of molecules, e.g., comprising at least about 50 different molecules, at least about 100 different molecules, at least about 200 different molecules, at least about 500 different molecules, at least about 1000 different molecules, at least about 5000 different molecules, at least about 10,000 molecules, etc.

The term “genome” refers to all nucleic acid sequences (coding and non-coding) and elements present in any virus, single cell (prokaryote and eukaryote) or each cell type in a metazoan organism. The term genome also applies to any naturally occurring or induced variation of these sequences that may be present in a mutant or disease variant of any virus or cell or cell type. Genomic sequences include, but are not limited to, those involved in the maintenance, replication, segregation, and generation of higher order structures (e.g., folding and compaction of DNA in chromatin and chromosomes), or other functions, if any, of nucleic acids, as well as all the coding regions and their corresponding regulatory elements needed to produce and maintain each virus, cell or cell type in a given organism.

For example, the human genome consists of approximately 3.0×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome Xs (female) for a total of 46 chromosomes. A genome of a cancer cell may contain variable numbers of each chromosome in addition to deletions, rearrangements and amplification of any subchromosomal region or DNA sequence. In certain aspects, a “genome” refers to nuclear nucleic acids, excluding mitochondrial nucleic acids; however, in other aspects, the term does not exclude mitochondrial nucleic acids. In still other aspects, the “mitochondrial genome” is used to refer specifically to nucleic acids found in mitochondrial fractions.

As used herein, a “test nucleic acid sample” or “test nucleic acids” refer to nucleic acids comprising sequences whose quantity or degree of representation (e.g., copy number) or sequence identity is being assayed. Similarly, “test genomic acids” or a “test genomic sample” refers to genomic nucleic acids comprising sequences whose quantity or degree of representation (e.g., copy number) or sequence identity is being assayed.

As used herein, a “reference nucleic acid sample” or “reference nucleic acids” refers to nucleic acids comprising sequences whose quantity or degree of representation (e.g., copy number) or sequence identity is known. Similarly, “reference genomic acids” or a “reference genomic sample” refers to genomic nucleic acids comprising sequences whose quantity or degree of representation (e.g., copy number) or sequence identity is known. A “reference nucleic acid sample” may be derived independently from a “test nucleic acid sample,” i.e., the samples can be obtained from different organisms or different cell populations of the sample organism. However, in certain embodiments, a reference nucleic acid is present in a “test nucleic acid sample” which comprises one or more sequences whose quantity or identity or degree of representation in the sample is unknown while containing one or more sequences (the reference sequences) whose quantity or identity or degree of representation in the sample is known. The reference nucleic acid may be naturally present in a sample (e.g., present in the cell from which the sample was obtained) or may be added to or spiked in the sample.

If a polynucleotide or probe “corresponds to” a chromosome, the polynucleotide usually contains a sequence of nucleic acids that is unique to that chromosome. Accordingly, a polynucleotide that corresponds to a particular chromosome usually specifically hybridizes to a labeled nucleic acid made from that chromosome, relative to labeled nucleic acids made from other chromosomes. Array features, because they usually contain surface-bound polynucleotides, can also correspond to a chromosome.

A “non-cellular chromosome composition” is a composition of chromosomes synthesized by mixing pre-determined amounts of individual chromosomes. These synthetic compositions can include selected concentrations and ratios of chromosomes that do not naturally occur in a cell, including any cell grown in tissue culture. Non-cellular chromosome compositions may contain more than an entire complement of chromosomes from a cell, and, as such, may include extra copies of one or more chromosomes from that cell. Non-cellular chromosome compositions may also contain less than the entire complement of chromosomes from a cell.

A “CGH array” or “aCGH array” refers to an array that can be used to compare DNA samples for relative differences in copy number. In general, an aCGH array can be used in any assay in which it is desirable to scan a genome with a sample of nucleic acids. For example, an aCGH array can be used in location analysis as described in U.S. Pat. No. 6,410,243, the entirety of which is incorporated herein and thus can also be referred to as a “location analysis array” or an “array for CHIP-chip analysis.” In certain aspects, a CGH array provides probes for screening or scanning a genome of an organism and comprises probes from a plurality of regions of the genome. In one aspect, the array comprises probe sequences for scanning an entire chromosome arm, wherein probes targets are separated by at least about 500 bp, at least about 1 kb, at least about 5 kb, at least about 10 kb, at least about 25 kb, at least about 50 kb, at least about 100 kb, at least about 250 kb, at least about 500 kb and at least about 1 Mb. In another aspect, the array comprises probes sequences for scanning an entire chromosome, a set of chromosomes, or the complete complement of chromosomes forming the organism's genome. By “resolution” is meant the spacing on the genome between sequences found in the probes on the array. In some embodiments (e.g., using a large number of probes of high complexity) all sequences in the genome can be present in the array. The spacing between different locations of the genome that are represented in the probes may also vary, and may be uniform, such that the spacing is substantially the same between sampled regions, or non-uniform, as desired. An assay performed at low resolution on one array, e.g., comprising probe targets separated by larger distances, may be repeated at higher resolution on another array, e.g., comprising probe targets separated by smaller distances.

The probes on the microarray, in certain embodiments have a nucleotide length in the range of at least 30 nucleotides to 200 nucleotides, or in the range of at least about 30 to about 150 nucleotides. In other embodiments, at least about 50% of the polynucleotide probes on the solid support have the same nucleotide length, and that length may be about 60 nucleotides.

In one aspect, probes represent sequences from an organism such as Drosophila melanogaster, Caenorhabditis elegans, yeast, bird, fish, a mouse, a rat, a domestic animal, a companion animal, a primate, a human, etc. In certain aspects, probes representing sequences from different organisms are provided on a single substrate, e.g., on a plurality of different arrays.

A “CGH assay” using an aCGH array can be generally performed as follows. In one embodiment, a population of nucleic acids contacted with an aCGH array comprises at least two sets of nucleic acid populations, which can be derived from different sample sources. For example, in one aspect, a target population contacted with the array comprises a set of target molecules from a reference sample and from a test sample. In one aspect, the reference sample is from an organism having a known genotype and/or phenotype, while the test sample has an unknown genotype and/or phenotype or a genotype and/or phenotype that is known and is different from that of the reference sample. For example, in one aspect, the reference sample is from a healthy patient while the test sample is from a patient suspected of having cancer or known to have cancer.

In one embodiment, a target population being contacted to an array in a given assay comprises at least two sets of target populations that are differentially labeled (e.g., by spectrally distinguishable labels). In one aspect, control target molecules in a target population are also provided as two sets, e.g., a first set labeled with a first label and a second set labeled with a second label corresponding to first and second labels being used to label reference and test target molecules, respectively.

In one aspect, the reference target molecules in a population are present at a level comparable to a haploid amount of a gene represented in the target population. In another aspect, the reference target molecules are present at a level comparable to a diploid amount of a gene. In still another aspect, the reference target molecules are present at a level that is different from a haploid or diploid amount of a gene represented in the target population. The relative proportions of complexes formed labeled with the first label vs. the second label can be used to evaluate relative copy numbers of targets found in the two samples.

In certain aspects, test and reference populations of nucleic acids may be applied separately to separate but identical arrays (e.g., having identical probe molecules) and the signals from each array can be compared to determine relative copy numbers of the nucleic acids in the test and reference populations.

In one embodiment, the invention provides a method of copying non-bacteriophage DNA using a T7-like DNA polymerase. The method can be used to label a sample of non-bacteriophage DNA and/or to increase the sensitivity of an assay by increasing the numbers of copies of a target DNA in a sample.

In certain aspects, the T7-like DNA polymerase is T7, a functional equivalent thereof, or an exonuclease-deficient form thereof. As referred to herein, in certain aspects, a functional equivalent of a T7 DNA polymerase is a polymerase that remains bound to a DNA molecule for at least about 500 bases, or at least about 1,000 bases, at least about 5,000 bases or at least about 7,000 bases, before dissociating under conditions normally used in a primer extension reaction. In certain aspects, a T7-like DNA polymerase has at least the activity of T7 DNA polymerase in terms of processivity, polymerization speed or strand-displacement activity and/or may have increased activity relative to T7 polymerase. In one aspect, a T7-like DNA polymerase can polymerize more than 70 kb in one binding event at a speed of 300 nt/sec.

A functional equivalent of a T7-like polymerase can include a homologous polymerase from another bacteriophage or a cell (e.g., a prokaryotic or eukaryotic cell) or recombinant forms thereof. Such a polymerase can further include one or more nucleotide modifications (e.g., insertions, deletions, fusions, and the like) that provide the polymerase with one or more of the activities of a T7 polymerase, and in certain aspects, at least the processivity of a T7 polymerase.

In certain aspects, a T7-like DNA polymerase has less than has less than 50%, less than 1%, and/or less than 0.1%, of the 3′ to 5′ exonuclease activity of T7 polymerase (i.e., which is typically, about 5,000 units of exonuclease activity per mg of polymerase—see, e.g., Chase et al. J Biol Chem. 1974;249:4545). In certain aspects, the T7-like polymerase comprises a polymerase sold commercially as T7 Sequenase version 2.0 (Tabor and Richardson, J Biol Chem. 1987;264:6647-6658; USB Corporation, Cleveland, Ohio).

In certain aspects, a T7-like DNA polymerase comprises a phage T7-encoded gene 5 protein (Modrich et al. J Biol Chem. 1975;150:5515) or a recombinant form thereof or a protein of identical sequence.

In one aspect, a T7-like polymerase has an error rate of 1.5×10⁵ or less.

In another aspect, a T7-like polymerase can initiate strand displacement at a nick in a double-stranded DNA template.

In certain aspects, in addition to having at least one of the activities described above, a T7-like polymerase according to aspects of the invention, is a thermostable protein, e.g., retains substantially all of its activity at greater than about 50° C., 60° C., 80° C., or 90° C.

As described further below, a T7-like DNA polymerase can further include a T7 DNA core polymerase (e.g., such as T7-encoded gene 5 or functional equivalents thereof and/or exonuclease deficient forms thereof) bound to accessory ptoteins to form a T7-like DNA holoenzyme. Such a holoenzyme can be reconstituted in vitro such that the proper stochiometry of proteins is obtained.

In certain embodiments, the method comprises contacting a sample of non-phage DNA with a T7-like polymerase in the presence of one or more accessory proteins, such as for example, thioredoxin, a helicase, a primase, a single stranded binding protein, and/or functionally equivalent proteins. In certain aspects, a single protein provides both helicase and primase activities, for example, an accessory protein such as gene 4 protein is provided.

Accessory proteins can include proteins encoded by the T7 genome and/or a bacterial genome (e.g., an E. coli genome) and/or recombinant forms thereof and/or functional equivalents thereof. Functional equivalents of accessory proteins are not necessarily encoded by T7 bacteriophage genomes and generally include any proteins that can promote one or more of the functional activities of a T7-like protein as described above, in vitro and/or in vivo. In certain aspects, such proteins physically and functionally interact with each other in vitro and in vivo substantially the same way the native proteins do.

For example, helicases can be encoded by bacterial genomes, T4 genomes, SV40 genomes (e.g., Large T antigen), yeast genomes (e.g., RAD) and other genomes and/or can be modified by mutation or recombinant DNA technology (e.g., by site directed mutagenesis or the production of chimeric forms and/or truncated forms) to provide functional equivalents of T7 accessory proteins.

A functional equivalent of an accessory protein can be readily identified by comparing its function to a protein encoded by a T7 genome, e.g., helicase, primase, and SSB, or E. coli genome, e.g., thioredoxin, and whose activity does not deviate significantly (as determined by routine statistical tests) from the activity of the protein encoded by the T7 genome or E. coli genome. In certain aspects, the activity measured is ability to stimulate replication of a T7 genome. In one aspect, the activity measured is the ability to bind to a protein (e.g., T7 polymerase) or a nucleic acid molecule with the similar binding properties (e.g., the binding properties of the functionally equivalent protein are not significantly different from those of the T7 accessory protein).

As used herein, a thioredoxin is protein that binds to a T7-like polymerase. In one aspect, a thioredoxin protein for use in methods of the invention is a protein encoded by an E. coli genome (Tabor et al., J. Biol, Chem. 1987;262:16, 216), or is a recombinant form thereof. In another aspect, a thioredoxin binds to the T7-like polymerase in a 1:1 stoichiometry. In still another aspect, a thioredoxin has a dissociation constant of about 5 nM. In a further aspect, binding of thioredoxin to a T7-like polymerase increases affinity of the T7-like polymerase for a primer-DNA template at least about 10-fold, at least about 50-fold, or at least about 80-fold.

As used herein a helicase is an enzyme that at least catalyzes the unwinding of a nucleic acid duplex. In one aspect, the helicase is encoded by T7 gene 4 or a recombinant form thereof.

As used herein, a primase is an enzyme that synthesizes RNA primers and permits T7 polymerase to extend RNA primers. In certain aspects, both helicase and primase functions are provided by the same protein, T7 gp4 (Richardson Cell 1983;33: 315-317). However, in certain aspects, primase is provided by a protein without helicase activity (see, e.g., Kato, et al. J Biol Chem., 2001; 276(24):21809-20).

As used herein, a T7 SSB protein or a functional equivalent thereof, enhances the unwinding activity of a T7 helicase and is a single stranded DNA binding protein. In one aspect, the T7 SSB protein is T7 gene 2.5 or a recombinant form thereof. In one aspect, a T7 SSB or functional equivalent thereof, binds to T7 gene 2.5 or a T7-like DNA polymerase, as described above. In another aspect, an SSB protein or functional equivalent thereof, increases the processivity of the T7-like protein, in one aspect, at least about 1000 fold. In certain aspects, an SSB protein or functional equivalent thereof, in conjunction with the T7-like polymerase, is utilized in a labeling and/or copying/amplification reaction in which a DNA template has regions of secondary structure.

Methods of purifying T7 accessory proteins are described, for example, in Kong and Richardson, J Biol Chem, 1988; 273(11):6556-6564. Accessory proteins can also be obtained by cloning using the DNA sequences for these proteins provided in GenBank. In certain aspects, recombinant accessory proteins can be overexpressed by plasmids carried within host cells and/or their expression can be controlled by cloning downstream of an inducible regulatory element such as a promoter.

One or all of the accessory proteins can be added to the sample of non-phage DNA and T7-like polymerase.

In certain embodiments, contacting of a T7-like polymerase and one or more accessory proteins to a sample of non-phage DNA is done in the presence of nucleotides, or modified or derivative forms thereof. In one aspect, the nucleotides are labeled. In another aspect, the nucleotides comprise all four of dATP, dTTP, dCTP, and dGTP or modified or derivative forms thereof. One or all of the nucleotides may be labeled with a detectable label. Labels include but are not limited to: fluorescent labels, chemiluminescent labels, and biotinylation. Other labeling methods, including radioactive isotopes, chromophores and biotin or hapten ligands, allow detection through the specific interaction with labeled molecules, like streptavidin and; antibodies. In certain aspects, labels include cyanine dyes, e.g., Cy3 and/or Cy5.

In certain aspects, the nucleotides are not chain-terminating nucleotides, e.g., the nucleotides do not include dideoxynucleotides.

In certain aspects, contacting is done in the presence of an oligonucleotide which is complementary to a subsequence of the non-bacteriophage DNA and/or which hybridizes to the subsequence of the non-bacteriophage DNA under stringent hybridization conditions. In another aspect, contacting is done in the presence of a plurality of oligonucleotides. In one aspect, the plurality is selected to bind randomly to subsequences of the non-bacteriophage DNA. The oligonucleotides can include random or degenerate sequences or can be designed to bind to a plurality of different known genomic locations on at least one strand of a genomic template.

Oligonucleotides can range from about 4-50 bases, or from about 6 to about 20 bases. In still other aspects, a sample of genomic template can be fragmented and linker sequences ligated to the termini of such fragments and oligonucleotides can be selected which are complementary to the linker sequences. In certain aspects, the oligonucleotides are labeled.

In still other aspects, the oligonucleotides are exonuclease resistant, i.e., modified so that they are not subject to exonuclease activity of the T7-like polymerase if the polymerase includes such activity. For example, the third base from the 3′ end of the oligonucleotide can include a ribonucleotide connected to the penultimate base through a phosphorothioate linkage. This modification is known to increase the half life of the oligonucleotide from 2 seconds to 18 minutes in a reconstitution assay (see, e.g., Griep and McHenry, J Biol Chem. 1990;265(33):20356-63). Alternatively, as described above, a T7-like DNA polymerase lacking 3′-5″ exonuclease activity can be used.

In general, while the DNA in a sample may be double-stranded, it can be converted into single-stranded or partially single stranded forms during at least a portion of the method. For example, the sample can be denatured by means of heating and/or exposure to a chemical agent. However, in certain aspects, the sample genomic DNA is not heated or exposed to a chemical agent to denature the strands. For example, the sample can be contacted with an accessory protein which is a strand-denaturing enzyme such as a helicase.

As discussed above, the sample DNA is a non-bacteriophage DNA, i.e., does not include substantial complementarity to a bacteriophage sequence over greater than 100 bases or over greater than 100 bases, though small regions of complementarity to bacteriophage DNA may be included (e.g., less than 500 or less than 100 bases). In certain aspects, the sample DNA comprises eukaryotic DNA, such as mammalian DNA and more particularly human DNA. In another aspect, the DNA is genomic DNA. Generally, the DNA is non-circular DNA (e.g., not plasmid DNA or mitochondrial DNA). In one aspect, the method excludes copying bacteriophage DNA, circular DNA (e.g., plasmid or mitochondrial DNA), cDNA or DNA with a complexity which is less than that of a bacterial genome, or less than that of a yeast genome. In certain aspects, the DNA sample has the complexity of at least an E. coli genome, an algal genome, a fungal genome, a fish genome, an avian genome, or a mammalian genome. Genome complexity can be determined using methods known in the art, e.g., such as by measuring Cₒt values.

The genomic source or sample may be prepared using any convenient protocol. In embodiments, the genomic source is prepared by obtaining a starting composition of genomic DNA (e.g., a cell lysate or a nuclear fraction thereof) where any convenient protocol or method for obtaining such a sample may be employed and numerous protocols for doing so are well known in the art. The genomic source is, in embodiments, genomic DNA representing the entire genome from a particular organism, tissue, or cell type.

A given initial genomic source may be prepared from a subject, for example a plant or an animal, that is suspected of being homozygous or heterozygous for a deletion or amplification of a genomic region. In an embodiment, the average size of the constituent molecules that make up the initial genomic source typically have an average size of at least about 1 Mb, where a representative range of sizes is from about 50 to 250 Mb or more, while in other embodiments, the sizes may not exceed about 1 MB, such that the may be about 1 Mb or smaller (e.g., less than about 500 Kb).

In certain aspects, the sample DNA is obtained from a formalin-fixed paraffin-embedded sample and/or from degraded or damaged fragmented genomic DNA. Fragment sizes can range from about 100 to about 1000 bases.

In one embodiment, genomic DNA is extracted and purified from biological tissues or clinical samples of interest.

In certain aspects, methods according to embodiments of the invention find particular use in applications where initially small sample volumes are to be analyzed. For example, small samples may be derived after purification of sub-populations of cells of interest (e.g., cells which have abnormal morphology) from a starting tissue sample. In addition, single and multi-parameter flow cytometry can identify small numbers of abnormal cells in a background of large numbers of normal cells in a biopsy or mixed cell population. Another technique that may be used to produce small samples of purified cells is laser capture microdissection (LCM). methods described in this application also find use where the samples are derived from complex tissues such as human biopsies that often contain elements such as, but not limited to, proteins, lipids, sugars and both organic and inorganic contaminants that inhibit replication and labeling of DNA templates derived from the tissues.

In embodiments of the invention, contacting is done under conditions in which the non-bacteriophage DNA is copied and/or amplified by the T7-like polymerase. In one aspect, the method comprises adding a T7-like polymerase, one or more accessory proteins, one or more nucleotide triphosphates (which are optionally labeled), adding a DNA sample which does not include bacteriophage DNA, adding appropriate oligonucleotide primer molecules as described above, and incubating the mixture at suitable temperatures to allow extension of the primer molecule(s). In certain aspects, conditions permit labeling and/or copying and/or amplifying of the template molecule. As used herein, the term “copying” generally encompasses amplification methods and the two terms may be used interchangeably herein.

In certain aspects, contacting occurs under isothermal conditions, e.g., temperature is not varied more than about 5° C., or more than about 2° C., or more than about 1° C. In one aspect, contacting occurs at room temperature, e.g., from about 21° C. to about 25° C. In certain aspects, isothermal conditions are preceded by exposing at least the DNA template to higher temperature conditions, e.g., to wholly or partially denature a double-stranded template. Additionally, or alternatively, isothermal conditions are terminated by contacting the reaction mix to a higher temperature, for example, to inactivate one or more proteins in the mix and/or to denature the replicated template.

Contacting can be carried out a temperature ranging from about 5° C. to about 40° C., or from about 15° C. to about 30° C., for a period of time ranging from about 1 hr to about 12 hr. However, aspects of the invention, include contacting for about 5 minutes to about an hour.

In certain aspects, after a time interval, the reaction mix is exposed to heat or other conditions to inactivate one or more proteins in the mix as described above. For example, the reaction mix can be heated to a temperature of about 50° C. to about 100° C. for a period of time ranging from about 1 min to about 10 min.

In certain aspects, oligonucleotide primers are contacted with a template under annealing conditions (e.g., generally after the template is rendered at least partially single stranded). In one aspect, primer annealing conditions include an annealing temperature of from about 20° C. to about 80° C., or from about 37° C. to about 65° C.

In certain embodiments, a “snap-cooling” protocol is employed, where the temperature is reduced to the annealing temperature, or to about 4° C. or below in a period of from about 1 s to about 30 s, usually from about 5 s to about 10 s after exposing template DNA to higher temperatures.

Primers can be contacted to the template prior to contacting the template with T7-like DNA polymerase and/or the one or more accessory proteins, or can be contacted at the same time or after the template is contacted with the T7-like polymerase and/or one or more accessory proteins.

In certain aspects, co-factors of accessory proteins are provided. For example, ATP, dATP, or dTTP can be provided as a co-factor for an accessory protein such as helicase. Suitable concentration ranges can include from 0.1-200 mM.

Methods according to the invention can be used to label and/or to copy and/or amplify DNA in a sample. In certain aspects, primers are provided which hybridize to both strands of a template DNA molecule; however, in other aspects, primers can be provided which hybridize to a single strand.

In certain aspects, primers on the same or opposite strands are separated by a distance of at least approximately, 100 nucleotides, 200 nucleotides, 500 nucleotides, 1,000 nucleotides, or even 2,000 nucleotides i.e., amplification or copied products of about base pairs, 500 base pairs, 1,000 base pairs, 2,000 base pairs, or 7,000 base pairs or more are produced.

Additional reagents that can be added to the reaction mix include, but are not limited to monovalent or divalent cations (e.g., Magnesium), DTT, EDTA, and the like. Other reagents such as polyethylene glycol, BSA, trehalose, or other carbohydrates, protein stabilizing agents, and the like can be added. In certain aspects, a surfactant such as Triton can also be added (e.g., 0.001-0.2%) The pH of the reaction mixture can range from 6-9 pH, and in certain cases may range from 6-8.

In certain embodiments, the methods are used to copy template DNA to be used for a genome-wide scanning application, such as CGH or location analysis.

In one embodiment, the invention provides methods for copying at least two samples of non-bacteriophage nucleic acids in the presence of first labeled nucleotides and second labeled nucleotides, respectively. In one aspect, the first labeled nucleotides are labeled with Cy3 while the second labeled nucleotides are labeled with Cy5. In another aspect, after the two samples are copied, copied nucleic acids are contacted to a support comprising nucleic acids, e.g., such as a chemical array substrate comprising a plurality of probe nucleic acids. In certain aspects, sample nucleic acids are initially copied (e.g., in the presence of unlabeled nucleotides) and the copied nucleic acids are then labeled (in the presence of labeled nucleotides). Copying and/or labeling can be done using T7-like DNA polymerase and one or more accessory proteins as described herein. However, in certain aspects, labeling is not performed.

For example, in certain embodiments, binding events on the surface of a substrate may be detected by methods other than by detection of a labeled probe nucleic acids, such as by change in conformation of a conformationally labeled immobilized target, detection of electrical signals caused by binding events on the substrate surface, and the like. In other embodiments, however, the populations of probe nucleic acids are labeled, where the populations may be labeled with the same label or different labels, depending on the actual assay protocol employed.

For example, where each population is to be contacted with different but identical arrays, each probe nucleic acid population or collection may be labeled with the same label. Alternatively, where both populations are to be simultaneously contacted with a single array of targets (i.e., co-hybridized to the same array of immobilized target nucleic acids) the populations are generally distinguishably or differentially labeled with respect to each other.

The two or more (i.e., at least first and second, where the number of different collections may, in certain embodiments, be three, four, or more) populations of probe nucleic acids are prepared from different genomic templates that are, in turn, prepared from different genomic sources.

In a further aspect, the first and second samples comprise test and reference nucleic acids, respectively, and the relative ratio of a target sequence in the first and second sample is determined, e.g., to evaluate the relative copy number of the target in the samples, for example, to determine the presence of duplications or deletions of the target in the test sample compared to the reference sample.

As such, embodiments of the disclosure may be used in methods of comparing abnormal nucleic acid copy number and mapping of chromosomal abnormalities associated with a disease. In embodiments, the methods may be employed in applications that use probe nucleic acids immobilized on a solid support (such as an array), to which differentially labeled target nucleic acids that are produced by using the T7-like polymerase and accessory proteins, are hybridized. Analysis of results of such experiments provides information about the relative copy number of nucleic acid regions (e.g., genes) in genomes. Variations in copy number detectable by CGH methods such as described above may arise in different ways. For example, the copy number may be altered as a result of amplification or deletion of a chromosomal region (e.g., as commonly occurs in cancer). Representative applications in which the CGH methods find use are further described in U.S. Pat. Nos. 6,335,167; 6,197,501; 5,830,645; and 5,665,549; the disclosures of which are herein incorporated by reference.

It should be noted that more than two genomic sources can be compared, but for reasons of clarity, only two genomic sources are described herein.

In still another embodiment, a sample of nucleic acids is bound to proteins from a cellular source, e.g., via crosslinking, and nucleic acids bound to protein(s) of interest are obtained (e.g., via immunoprecipitation) before or after a fragmentation step. Fragmentation may be achieved using any convenient protocol, including but not limited to: mechanical protocols (e.g., sonication, shearing, and the like) and chemical protocols (e.g., enzyme digestion, and the like). In certain aspects, fragmented molecules range in size from about 200 bp to about 10 Kb, or from about 1000 bp to about 10 Kb.

Binding of nucleic acids to the protein of interest is reversed (e.g., by heating) and the fragments are copied using a method as described above. In certain aspects, the fragments bound to the protein of interest and which have been copied are contacted to a chemical array. Methods of performing such location analysis are described in, for example, in U.S. Pat. No. 6,410,243.

Methods of labeling and/or copying DNA according to embodiments of the invention can also be used in comprehensive studies including genotyping (e.g., of single nucleotide polymorphisms (SNPs), copy number polymorphisms (CNPs), sequencing, and cDNA analyses).

Embodiments of the invention additionally include kits. In one aspect, a kit includes a T7-like polymerase, one or more accessory proteins, such as thioredoxin, a helicase, a primase, SSB and/or functional equivalents thereof. In certain aspects, helicase and primase activity are provided by a single protein. Proteins may be provided in solution (and optionally in the presence of protein stabilizing reagents) or can be provided in a lyophilized form. In certain aspects, a plurality of proteins are provided in a single solution or lyophilization mix. In other aspects, individual proteins are provided in separate solution containers or lyophilization mixes.

Co-factors, monovalent or divalent cations can be included in the kits as well as reagents such as DTT and/or EDTA. The kit may additionally include oligonucleotide primers and/or adaptors, ligase (e.g., to ligate adaptors to the termini of DNA fragments to be labeled, copied and/or amplified), a topoisomerase and/or nucleotides. The nucleotides may be labeled or unlabeled and can include for example, all four nucleotides such as DATP, dTTP, dCTP, dGTP. In certain aspects, the kit does not include a chain-terminating nucleotide such as a dideoxynucleotide. In certain aspects, the oligonucleotide primers are labeled. In certain aspects, reagents for labeling a nucleotide or primer are provided.

In additional aspects, the kit can include a control sample of genomic DNA and/or reagents for isolating genomic DNA, e.g., such as detergents, salts, buffers, and/or isolation columns or membranes.

In certain aspects, the kit can include a cross-linking agent such as paraformaldehyde, formaldehyde, glutaraldehyde or combinations thereof, antibodies or other binding molecules (e.g., aptamers, affibodies, antibody fragments and the like) which recognize DNA-binding proteins of interest (e.g., such as histones and/or associated proteins, transcription factors, centromere-binding proteins, telomere-binding proteins and the like). The kit may optionally include an agent for fragmenting DNA, such as a sonicator or one or more enzymes (e.g., a nuclease, a restriction enzyme and the like). The antibodies or other binding molecules may optionally be attached to a solid support.

In further aspects, the kits can include nucleic acids immobilized on a solid support. For example, one or more arrays can be provided on a single or multiple substrates.

In still further aspects, kits may include reagents for isolating nucleic acids from clinical samples, e.g., such as reagents for isolating nucleic acids from frozen or paraffin-embedded samples. Such reagents can include but are not limited to a solvent and/or other de-paraffininizing reagent, an alcohol, a chaotropic salt, and the like.

Finally, the kits may further include instructions for using the kit components in the subject methods. The instructions may be printed on a substrate, such as paper or plastic, etc. As such, the instructions may be present in the kits as a package-insert, in the labeling of the container, of the kit or components thereof (i.e., associated with the packaging or sub-packaging). In other embodiments, the instructions are present as an electronic storage data file present on a suitable computer readable storage medium (e.g., CD-ROM, diskette, and the like).

### PROPHETIC EXAMPLE

Reference is now made to the following example, which together with the above description, illustrates the invention in a non-limiting fashion.

Exemplary prophetic protocols that can be used for preparing samples by a T7-like polymerase for subsequent analysis (e.g., CGH, location analysis, and the like) are described below. The protocols can be used for both labeling and amplification of genomic DNA (gDNA).

To generate amplified target molecules from gDNA, a T7-like DNA polymerase can be reconstituted in vitro by adding recombinant accessory proteins and SSB. For example, T7 DNA polymerase can be reconstituted in vitro by adding recombinant T7 gp5 and a saturated amount of recombinant thioredoxin (trx) followed by purification to remove excess proteins to get a 1:1 gp5/TRX complex (see, e.g., as described in Johnson and Richardson, J Biol Chem. 2003;278(26):23762-72). In the description below, the term “T7-polymerase” is used to refer to a T7 gp5/TRC complex unless otherwise indicated. Alternatively, T7 DNA polymerase can be reconstituted by adding gp4, which has both primase and helicase activity or by adding a modified gp4 protein in which primase activity is removed by chemical or genetic modification (i.e., such that the protein only has helicase activity) and adding random primers. SSB can be added to promote enzyme function. Labeled nucleotides can be added initially (e.g., one or more of the nucleotides: DATP, dTTP, dCTP, or dGTP can be labeled) or after one or more initial rounds of amplification/copying of the gDNA template. Alternatively, the copied template can be digested with restriction enzymes or nucleases prior to labeling with the T7 DNA polymerase-like enzyme. In certain aspects, digested gDNA is purified by an appropriate DNA purification method. The purified digested gDNA is denatured by heat or alkaline denaturation for primer annealing and subsequent contacting by T7 polymerase and one or more accessory proteins as described above.

A 50 μl reaction containing 6 μg of purified, digested genomic DNA, 10 nmol of primer, 13 units of T7 DNA polymerase (gp5/TRX complex) (1 unit=incorporation of 1 nmol of acid soluble dNTPS to acid insoluble forms at 37° C. for 30 sec), 50 mM Tris-Cl, pH 7.5, 10 mM MgCl₂, 0.1 mM MnCl₂, 0.1 mM DTT, 50 mM NaCl, 500 μM dATP, dGTP, dCTP, 100 μM of dTTP and 100 μM of labeled TTP (typically includes, but is not limited to—fluorophore, radioisotope, or biotin-conjugated deoxynucleotides), and 10 μg of SSB is incubated at 37° C. for 10 minutes. The ratio of gDNA amount and primer can be optimized by titration experiments. Similarly, the optimum SSB amount per gDNA can be determined by titration.

Additionally, the ratio of Mg⁺⁺ and Mn⁺⁺ concentrations can be optimized by titration experiments to enhance the incorporation of nucleotides (e.g., labeled nucleotides), while still maintaining the high fidelity of the T7-like polymerase. Other divalent ions can be used for similar effect.

The reaction can be stopped by addition of EDTA to a final concentration of 25 mM or by incubation at 70° C. for 5 minutes. The scale of the reaction can be adjusted (i.e., scaling up or down) to yield larger amounts of product for a given application.

Labeled targets can be fragmented, denatured or further treated to enable quantitative, qualitative, and reproducible detection by analytical instruments such as a laser scanner or an Agilent 2100 bioanalyzer device. However, such steps are optional.

The hybridization method and stringencies can be optimized to be adequate for an application, e.g., such as an existing array platform for genomic hybridization assays. For example, for a microarray containing high levels of GC, the hybridization method should be optimized to enable temperatures that are compatible with the existing array platform.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the invention.

What is claimed is:

1. A method comprising:
contacting a sample of non-bacteriophage, non-circular, genomic DNA with a T7-like polymerase in the presence of at least one accessory protein, an oligonucleotide capable of binding to a sequence of the non-bacteriophage genomic DNA, and one or more nucleotides, under conditions wherein the oligonucleotide binds to the sequence of the non-bacteriophage genomic DNA and the T7-like polymerase extends the primer.

2. The method of claim 1, wherein the at least one accessory protein is selected from the group consisting of a thioredoxin, a helicase, a primase, a single-stranded binding protein, functionally equivalent proteins, and combinations thereof.

3. The method of claim 1, wherein the at least one accessory protein is obtained by overexpressing a recombinant form of the protein in a host cell.

4. The method of claim 1, wherein the method further comprises reconstituting a T7-like DNA polymerase holoenzyme in vitro.

5. The method of claim 1, wherein contacting is done in the presence of a thioredoxin, a helicase, a primase, and a single-stranded binding protein.

6. The method of claim 2, wherein helicase and primase activities are provided in a single protein.

7. The method of claim 1, wherein the sample of genomic DNA has the complexity of at least an E. coli genome.

8. The method of claim 1, wherein the sample of genomic DNA has the complexity of a mammalian genome.

9. The method of claim 1, wherein contacting occurs in the presence of a plurality of random or degenerate sequence oligonucleotides.

10. The method of claim 1, wherein at least one of the one or more nucleotides is labeled.

11. The method of claim 1, wherein the contacting occurs under conditions suitable for copying the genomic DNA in the sample.

12. The method of claim 1, wherein the contacting occurs under conditions suitable for labeling the genomic DNA in the sample.

13. The method of claim 12, wherein the contacting occurs under conditions suitable for labeling copied genomic DNA.

14. The method of claim 1, further comprising the step of fragmenting the genomic DNA.

15. The method of claim 14, wherein the fragmenting is performed by contacting the genomic DNA with a nuclease.

16. The method of claim 1, further comprising the step of contacting primer extension products to an array.

17. The method of claim 1, further comprising performing said method on first and second separate samples and mixing primer extension products.

18. The method of claim 17, wherein the primer extension products from the first and second samples are differentially labeled.

19. The method of claim 17, comprising determining relative amounts of at least one sequence in the first and second samples.

20. The method of claim 1, further comprising performing said method on first and second separate samples and contacting primer extension products to the same array or to at least two arrays comprising at least a subset of identical sequences at features of the arrays.

21. The method of claim 1, wherein the genomic sample comprises DNA binding proteins bound thereon and wherein the method comprises a fragmentation step to fragment the genomic DNA at sequences not bound by the DNA binding proteins.

22. The method of claim 21, wherein the DNA binding proteins are crosslinked to the genomic DNA.

23. The method of claim 21, further comprising obtaining DNA fragments bound to a DNA binding protein of interest prior to the contacting step.

24. The method of claim 23, wherein the obtaining comprises an immunoprecipitation step.

25. The method of claim 23, wherein the method further comprises obtaining primer extension products and contacting the products to an array.

26. A method comprising:
contacting a test sample and a reference sample of genomic DNA with a T7-like polymerase in the presence of at least one accessory protein, an oligonucleotide primer capable of binding to a sequence of the genomic DNA, and one or more nucleotides, under conditions wherein the oligonucleotide primer binds to the sequence of the genomic DNA in the test and reference samples and the T7-like polymerase extends the primer,
obtaining primer extension products from the first and second samples and contacting primer extension products to the same array or to at least two arrays comprising at least a subset of identical sequences at features of the arrays.

27. The method of claim 26, further comprising determining relative amounts of at least one sequence in the test and reference sample.

28. A method comprising:
contacting a sample of genomic DNA that comprises DNA binding proteins bound thereon;
fragmenting the genomic DNA at sequences not bound by the DNA binding proteins;
obtaining DNA fragments bound to a DNA binding protein of interest;
removing the DNA binding protein of interest from the DNA fragments;
contacting the DNA fragments with a T7-like polymerase in the presence of at least one accessory protein, oligonucleotide primers capable of binding to a sequence of a plurality of the fragments, and one or more nucleotides, under conditions wherein the oligonucleotides binds to the sequence of the fragments and T7-like polymerase extends the primer, and contacting primer extension products to an array of nucleic acids.

29. The method of claim 28, further comprising determining the location and/or sequence of a fragment to which the DNA binding protein of interest binds.

30. A kit comprising a T7-like polymerase, at least one accessory protein, and a sample of non-bacteriophage, non-circular genomic DNA.

31. The kit of claim 30, wherein the sample comprises genomic DNA having at least the complexity of E. coli DNA.

32. The kit of claim 30, wherein the sample comprises genomic DNA having at least the complexity of mammalian DNA.

33. A kit comprising a T7-like polymerase, at least one accessory protein, and random or degenerate sequence oligonucleotides for binding to a plurality of genomic DNA sequences, and nucleotides labeled with spectrally distinguishable labels.

34. A kit comprising a T7-like polymerase, at least one accessory protein, and a deparaffinizing reagent.

35. A kit comprising a comprising a T7-like polymerase, at least one accessory protein, and an antigen-binding molecule specific to a DNA binding protein.
